(19) United States
(12) Patent Application Publication
(10) Pub. No.: US 2023/0097574 A1
(43) Pub. Date: Mar. 30, 2023

(54) METHODS, SYSTEMS, AND COMPUTER PROGRAM PRODUCTS FOR AUTOMATICALLY SELECTING A NETWORK SOURCE FOR RETRIEVING CLINICAL INFORMATION

(71) Applicant: Change Healthcare Holdings LLC, Nashville, TN (US)

(72) Inventors: Arien Malec, Oakland, CA (US); Stephen Dean, Henrico, NC (US); Faisal Mushtaq, West Harrison, NY (US)

(21) Appl. No.: 17/449,329

(22) Filed: Sep. 29, 2021

(51) Int. Cl. G16H 10/60; G06Q 30/02; G06Q 40/08

(52) U.S. Cl. CPC G16H 10/60; G06Q 30/0283; G06Q 40/08

(57) ABSTRACT

A method includes receiving a request for clinical information associated with one or more patients from a requestor, the request being associated with requested values for one or more parameters associated with a plurality of network sources for retrieving the clinical information; comparing the requested values with characteristic values for the one or more parameters of each of the plurality of network sources to generate a comparison result; selecting one of the plurality of network sources based on the comparison result; and communicating the request for clinical information to the one of the plurality of network sources that was selected.

### FIELD

The present inventive concepts relate generally to health care systems and services and, more particularly, to retrieving clinical information associated with patients.

### BACKGROUND

Health care service providers record clinical information associated with patients under their care in clinical charts, which are typically stored as electronic health records. Various stakeholders may seek access to clinical information associated with patients for a variety of different reasons. These stakeholders may include, but are not limited to, payors (e.g., private and/or public health insurance entities), life insurance companies, and/or clinical research organizations. Various options may exist for a requestor to retrieve clinical information associated with one or more patients including different media types (e.g., paper or electronic), different data formats and/or protocols used for retrieving the clinical information, different network paths, including use of one or more intermediaries (e.g., use of a third party record retrieval service), different repositories for the clinical information (e.g., storage at the health care provider facility, storage at a cloud location, storage at a third-party managed facility, etc.). Typically, a requestor relies on the same method or service for retrieving patient clinical information, but this method may not always be the most cost effective and/or efficient in retrieving the clinical information the requestor desires.

### SUMMARY

According to some embodiments of the inventive concept, a method comprises: receiving a request for clinical information associated with one or more patients from a requestor, the request being associated with requested values for one or more parameters associated with a plurality of network sources for retrieving the clinical information; comparing the requested values with characteristic values for the one or more parameters of each of the plurality of network sources to generate a comparison result; selecting one of the plurality of network sources based on the comparison result; and communicating the request for clinical information to the one of the plurality of network sources that was selected.

In other embodiments, at least one of the requested values is received with the request for the clinical information.

In other embodiments, the method further comprises: determining whether a result of the request that was communicated to the one of the plurality of network sources was successful or unsuccessful; determining a financial cost associated with the request that was communicated to the one of the plurality of network sources; and updating the characteristic values of the one of the plurality of network sources based on the result of the request and the financial cost associated with the request.

In still other embodiments, the one or more parameters associated with the plurality of network sources comprise: a financial cost for request fulfillment; a success rate for request fulfillment; a media type used for request fulfillment; a media access type used for request fulfillment; a response time for request fulfillment; a document processing technique applied for request fulfillment; or purposes of use supported for request fulfillment.

In still other embodiments, the media type used for request fulfillment parameter is configured to assume a value of electronic format or paper format.

In still other embodiments, the media access type used for request fulfillment parameter is configured to assume one or more values of Fast Healthcare Interoperability Resources (FHIR) protocol, Electronic Data Interchange (EDI) format, United States Core Data for Interoperability (USCDI) Consolidated-Clinical Document Architecture (C-CDA) format, paper communication and/or electronic mail messaging.

In still other embodiments, the financial cost for request fulfillment is configured to be expressed as a financial cost for a single request for clinical information for a single patient; and/or the financial cost is configured to be expressed as a financial cost for multiple requests for clinical information of a single patient over a defined time period.

In still other embodiments, the defined time period is one year or one month.

In still other embodiments the financial cost for request fulfillment is dependent on values for the media type used for request fulfillment parameter and/or the media access type used for request fulfillment parameter.

In still other embodiments, the purposes of use supported for request fulfillment is configured to assume a value of benefits determination, life insurance eligibility determination, clinical research, or patient risk adjustment for insurance.

In still other embodiments, the method further comprises defining priority weights for the one or more parameters associated with the plurality of network sources. Selecting the one of the plurality of network sources based on the comparison result comprises selecting the one of the plurality of network sources based on the comparison result and the priority weights for the one or more parameters associated with the plurality of network sources.

In some embodiments of the inventive concept, a system comprises a processor; and a memory coupled to the processor and comprising computer readable program code embodied in the memory that is executable by the processor to perform operations comprising: receiving a request for clinical information associated with one or more patients from a requestor, the request being associated with requested values for one or more parameters associated with a plurality of network sources for retrieving the clinical information; comparing the requested values with characteristic values for the one or more parameters of each of the plurality of network sources to generate a comparison result; selecting one of the plurality of network sources based on the comparison result; and communicating the request for clinical information to the one of the plurality of network sources that was selected.

In further embodiments, the operations further comprise: determining whether a result of the request that was communicated to the one of the plurality of network sources was successful or unsuccessful; determining a financial cost associated with the request that was communicated to the one of the plurality of network sources; and updating the characteristic values of the one of the plurality of network sources based on the result of the request and the financial cost associated with the request.

In still further embodiments, the one or more parameters associated with the plurality of network sources comprise: a financial cost for request fulfillment; a success rate for request fulfillment; a media type used for request fulfillment; a media access type used for request fulfillment; a response time for request fulfillment; a document processing technique applied for request fulfillment; or purposes of use supported for request fulfillment; wherein the media type used for request fulfillment parameter is configured to assume a value of electronic format or paper format; wherein the media access type used for request fulfillment parameter is configured to assume one or more values of Fast Healthcare Interoperability Resources (FHIR) protocol, Electronic Data Interchange (EDI) format, United States Core Data for Interoperability (USCDI) Consolidated-Clinical Document Architecture (C-CDA) format, paper communication and/or electronic mail messaging; wherein the financial cost for request fulfillment is configured to be expressed as a financial cost for a single request for clinical information for a single patient, and/or wherein the financial cost is configured to be expressed as a financial cost for multiple requests for clinical information of a single patient over a defined time period; wherein the financial cost for request fulfillment is dependent on values for the media type used for request fulfillment parameter and/or the media access type used for request fulfillment parameter; and wherein the purposes of use supported for request fulfillment is configured to assume a value of benefits determination, life insurance eligibility determination, clinical research, or patient risk adjustment for insurance.

In still further embodiments, the defined time period is one year or one month.

In still further embodiments, the operations further comprise: defining priority weights for the one or more parameters associated with the plurality of network sources. Selecting the one of the plurality of network sources based on the comparison result comprises selecting the one of the plurality of network sources based on the comparison result and the priority weights for the one or more parameters associated with the plurality of network sources.

In some embodiments, a computer program product comprises a non-transitory computer readable storage medium comprising computer readable program code embodied in the medium that is executable by a processor to perform operations comprising: receiving a request for clinical information associated with one or more patients from a requestor, the request being associated with requested values for one or more parameters associated with a plurality of network sources for retrieving the clinical information; comparing the requested values with characteristic values for the one or more parameters of each of the plurality of network sources to generate a comparison result; selecting one of the plurality of network sources based on the comparison result; and communicating the request for clinical information to the one of the plurality of network sources that was selected.

In other embodiments, the operations further comprise: determining whether a result of the request that was communicated to the one of the plurality of network sources was successful or unsuccessful; determining a financial cost associated with the request that was communicated to the one of the plurality of network sources; and updating the characteristic values of the one of the plurality of network sources based on the result of the request and the financial cost associated with the request.

In still other embodiments, the one or more parameters associated with the plurality of network sources comprise: a financial cost for request fulfillment; a success rate for request fulfillment; a media type used for request fulfillment; a media access type used for request fulfillment; a response time for request fulfillment; a document processing technique applied for request fulfillment; or purposes of use supported for request fulfillment; wherein the media type used for request fulfillment parameter is configured to assume a value of electronic format or paper format; wherein the media access type used for request fulfillment parameter is configured to assume one or more values of Fast Healthcare Interoperability Resources (FHIR) protocol, Electronic Data Interchange (EDI) format, United States Core Data for Interoperability (USCDI) Consolidated-Clinical Document Architecture (C-CDA) format, paper communication and/or electronic mail messaging; wherein the financial cost for request fulfillment is configured to be expressed as a financial cost for a single request for clinical information for a single patient, and/or wherein the financial cost is configured to be expressed as a financial cost for multiple requests for clinical information of a single patient over a defined time period; wherein the financial cost for request fulfillment is dependent on values for the media type used for request fulfillment parameter and/or the media access type used for request fulfillment parameter; and wherein the purposes of use supported for request fulfillment is configured to assume a value of benefits determination, life insurance eligibility determination, clinical research or patient risk adjustment for insurance.

In still other embodiments, the defined time period is one year or one month.

In still other embodiments, the operations further comprise: defining priority weights for the one or more parameters associated with the plurality of network sources. Selecting the one of the plurality of network sources based on the comparison result comprises selecting the one of the plurality of network sources based on the comparison result and the priority weights for the one or more parameters associated with the plurality of network sources.

Other methods, systems, articles of manufacture, and/or computer program products according to embodiments of the inventive concept will be or become apparent to one with skill in the art upon review of the following drawings and detailed description. It is intended that all such additional systems, methods, articles of manufacture, and/or computer program products be included within this description, be within the scope of the present inventive subject matter and be protected by the accompanying claims.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram that illustrates a communication network including a system for automatically selecting a network source for retrieving clinical information in accordance with some embodiments of the inventive concept;

FIGS. 2A, 2B, 2C, and 2D are block diagrams that illustrate network sources for clinical information in accordance with various embodiments of the inventive concept;

FIG. 3 is a block diagram that illustrates the network source selection system according to some embodiments of the inventive concept;

FIG. 4-6 are flowcharts that illustrate operations of automatically selecting a network source for retrieving clinical information in accordance with some embodiments of the inventive concept;

FIG. 7 is a data processing system that may be used to implement a network source selection system for retrieving clinical information in accordance with some embodiments of the inventive concept; and

FIG. 8 is a block diagram that illustrates a software/hardware architecture for use in a network source selection system for retrieving clinical information in accordance with some embodiments of the inventive concept.

### DETAILED DESCRIPTION

In the following detailed description, numerous specific details are set forth to provide a thorough understanding of embodiments of the inventive concept. However, it will be understood by those skilled in the art that embodiments of the inventive concept may be practiced without these specific details. In some instances, well-known methods, procedures, components, and circuits have not been described in detail so as not to obscure the inventive concept. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “provider” may mean any person or entity involved in providing health care products and/or services to a patient.

As used herein, the term “network source” may mean a combination of network path and information repository. Thus, one network source may differ from another network source if they have different network paths, different information repositories, and/or different network paths and different information repositories.

Some embodiments of the inventive concept stem from a realization that stakeholders, such as payors (e.g., private and/or public health insurance entities), life insurance companies, clinical research organizations, and the like may seek access to patient clinical information stored, for example, as patient health records or charts. Oftentimes, the clinical information requested may be obtained from more than one information repository and/or using more than one network path. These clinical information repositories and/or network paths may have advantages and disadvantages relative to each other with respect to financial cost, convenience, reliability, speed, or other performance metrics. Embodiments of the inventive concept may provide a network source selection system that may receive a request for clinical information along with one or more values for parameters associated with the different network sources that may be used to retrieve the requested clinical information. These parameters may include, but are not limited to, a financial cost for fulfilling the request, a success rate in fulfilling requests, a media type used in fulfilling requests (e.g., electronic format or paper format), a media access type used in fulfilling requests (e.g., use of the Fast Healthcare Interoperability Resources (FHIR) protocol, use of the Electronic Data Interchange (EDI) format, use of the United States Core Data for Interoperability (USCDI) Consolidated-Clinical Document Architecture (C-CDA) format, use of paper communication and/or electronic mail messaging), a response time for request fulfillment (e.g., is the document requested in real time, near real time, over a 24-hour period, or some other requested response time), a document processing technique applied for request fulfillment (e.g., is the document pre-processed for duplication, standardization, and/or normalization), and the purposes of use supported in fulfilling requests.

The financial cost may be expressed in different ways. For example, the financial cost for request fulfillment may be expressed as a financial cost for a single request as some network sources charge a particular amount for each record pull corresponding to a single patient. The financial cost for request fulfillment may also be expressed as a financial cost for multiple requests for clinical information for a single patient over a defined time period, such as a single year or single month. In this regard, a network source may charge a requestor, such as a payor, a set amount based on a per member (e.g., patient) per year or month basis.

The purposes of use parameter may assume a value of benefits determination, life insurance eligibility determination, patient risk adjustment for insurance, and/or clinical research. Certain network sources may restrict the release of clinical information to requestors based on the manner in which the information will be used.

A requestor may define priority weights for the one or more parameters associated with the network sources. The network source selection system may use these weights in selecting one of the network sources to fulfill a request. For example, while one network source may provide the lowest financial cost, the network source may have a relatively low success rate for request fulfillment. A requestor may place greater priority on reliability as manifest by successful request fulfillments relative to financial cost. A network source may also provide a low financial cost, but is capable of fulfilling requests only in paper format resulting in a relatively slow response time and a more difficult format in which to review the retrieved clinical information. Accordingly, a requestor may place greater priority on media type relative to financial cost.

Once a network source is selected and the clinical information request is communicated to the network source, the network source selection system may determine whether the request was fulfilled successfully and may also determine the financial cost associated with fulfilling the request to update characteristic values of the parameters corresponding to the selected network source. The characteristic values may be a typical value or a range of values for one or more of the parameters corresponding to a particular network source.

The network source selection system may, therefore, identify a network source for fulfilling a request for clinical information that aligns with the requestor's preferences in terms of financial cost, convenience, format, and reliability.

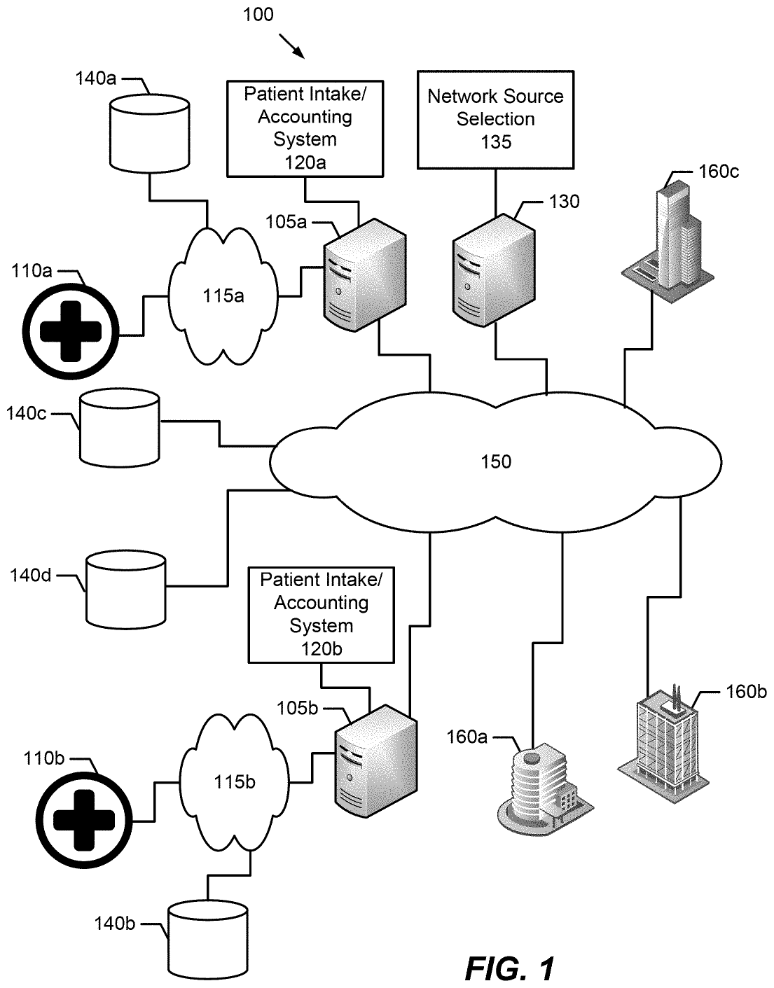

Referring to FIG. 1, a communication network 100 including a network source selection system for retrieving clinical information, in accordance with some embodiments of the inventive concept, comprises multiple health care provider facilities or practices 110a, 110b. Each health care provider facility or practice may represent various types of organizations that are used to deliver health care services to patients via health care professionals, which are referred to generally herein as “providers.” The providers may include, but are not limited to, hospitals, medical practices, mobile patient care facilities, diagnostic centers, lab centers, pharmacies, and the like. The providers may operate by providing health care services for patients and then invoicing one or more payors 160a for the services rendered. The payor 160a may be, but is not limited to, a provider of private insurance plans, a provider of a government insurance plan (e.g., Medicare, Medicaid, state, or federal public employee insurance plans), a provider of a hybrid insurance plan (e.g., Affordable Care Act plans), a provider of a private medical cost sharing plan, and the patient themself. Two provider facilities 110a, 110b are illustrated in FIG. 1 with the first provider including a first patient intake/accounting system server 105a accessible via a network 115a. The first patient intake/accounting system server 105a may be configured with a patient intake/accounting system module 120a to manage the intake of patients for appointments and to generate invoices for payors for services and products rendered through the provider 110a. The network 115a communicatively couples the first patient intake/accounting system server 105a to other devices, terminals, and systems in the provider's facility 110a. The network 115a may comprise one or more local or wireless networks to communicate with first patient intake/accounting system server 105a when the first patient intake/accounting system server 105a is located in or proximate to the health care service provider facility 110a. When the first patient intake/accounting system server 105a is in a remote location from the health care facility, such as part of a cloud computing system or at a central computing center, then the network 115a may include one or more wide area or global networks, such as the Internet. The second provider facility 110b is similar to the first provider facility 110a and includes a second patient intake/accounting system server 105b, which is configured with a patient intake/accounting system module 120b. The second patient intake/accounting system server 105b is coupled to other devices, terminals, and systems in the provider's facility 110b via a network 115b.

According to embodiments of the inventive concept, a network source selection system may be configured to retrieve clinical information by way of patient charts or records for one or more requestors, such as, but not limited to, a payor 160a, a life insurance provider 160b, and a clinical researcher 160c. The network source selection system includes a network source selection server 130, which may include a network source selection module 135 that may be configured to receive a request for clinical information along with one or more values for parameters associated with the different network sources that may be used to retrieve the requested clinical information. The clinical information that is requested may be obtained from more than one information repository and/or using more than one network path. For example, as shown in FIG. 1, clinical information repositories 140a, 140b are associated with providers 110a and 110b and may be accessed through their respective networks 115a and 115b. These providers 110a, 110b may also store their patient charts and records in other repositories, 140c and/or 140d, which are located in other network locations, such as in the cloud. These repositories 140c and 140d may represent a variety of different types of clinical information record repositories including, but not limited to, repositories managed by one or more third parties, repositories that are part of a national network, repositories that are part of an Electronic Health Record (EHR) network, and the like. Moreover, these clinical information repositories 140a, 140b, 140c, and 140d may be accessible by a variety of different network paths, which may have advantages and disadvantages relative to each other. For example, one network may provide a high speed link for retrieving clinical information, but at a relatively high financial cost. Another network may provide a lower speed link for retrieving clinical information, but at a relatively low financial cost. In addition, some network sources may have purpose of use limitations that limit access to information based on requestors that are authorized for a particular purpose of use. Moreover, a network source may not support all purposes of use. Thus, each network source may have one or multiple permitted purposes of use. And for those purpose(s) of use supported by a network source, requestors may need to be authorized or approved for a particular purpose of use. For example, a network source may support a direct connection to a clinical information repository only for those requestors that qualify for a particular purpose of use, e.g., operations in a medical center. These requestors may have a profile setup with the network source that grants them a particular purpose of use status for making clinical information requests. Such profiles may be generated, for example, through agreements or contracts between requestors and network sources. The network source selection module 135 may be configured to process the request for clinical information received from a requestor along with the one or more parameter values associated with the different network sources to select one of the network sources for retrieving the requested clinical information. The network source selection module 135 may weigh these tradeoffs between various parameters associated with the network sources based on defined weights for the parameters provided by, for example, the requestor. For example, a requestor may submit a request for clinical information with a media type format parameter value of electronic, a media access type parameter value of the FHIR protocol operating at greater than 500 Mbps, and a financial cost parameter value of less than $5 per patient record. The network source selection module 135 may select a network source for the clinical information based on these parameter values and the characteristic values for these parameters of the various network sources. The requestor may place greater priority on the media access parameter than the financial cost parameter by assigning a higher priority weight to the medica access parameter than the financial cost parameter. Thus, the network source selection module 135 may select a first network source that uses the FHIR protocol and operates at speeds greater than 500 Mbps over a second network source that uses the FHIR protocol and operates at only 200 Mbps even though the second network source charges $3 per patient record and the first network source charges $7 per patient record, because the requestor has placed a priority on speed in obtaining the clinical information.

A network 150 couples the patient intake/accounting system servers 105a, 105b to the network source selection server 130 and couples requestors, such as the payor 160a, life insurance provider and 160b, and clinical researcher 160c to the network source selection server 130 and the various network sources for retrieving clinical information. The network 150 may be a global network, such as the Internet or other publicly accessible network. Various elements of the network 150 may be interconnected by a wide area network, a local area network, an Intranet, and/or other private network, which may not be accessible by the general public. Thus, the communication network 150 may represent a combination of public and private networks or a virtual private network (VPN). The network 150 may be a wireless network, a wireline network, or may be a combination of both wireless and wireline networks.

The network source selection service for retrieving clinical information provided through the network source selection server 130 and the network source selection module 135 may, in some embodiments, be embodied as a cloud service. For example, clinical information requestor may access the network source selection system as a Web service. In some embodiments, the network source selection system service may be implemented as a Representational State Transfer Web Service (RESTful Web service).

Although FIG. 1 illustrates an example communication network including a network source selection service for automatically selecting a network source for retrieving clinical information, it will be understood that embodiments of the inventive subject matter are not limited to such configurations, but are intended to encompass any configuration capable of carrying out the operations described herein.

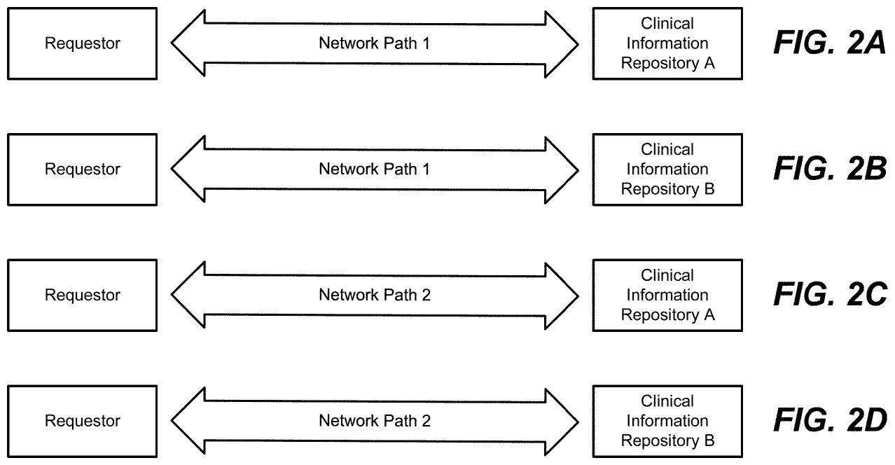

FIGS. 2A, 2B, 2C, and 2D are block diagrams that illustrate different network sources for retrieving clinical information therefrom in accordance with various embodiments of the inventive concept. As shown in FIGS. 2A-2D, a network source is defined as a combination of a network path and a clinical information repository. Network sources differ from one another with respect to the network path used and/or the clinical information repository used. For example, as shown in FIGS. 2A and 2C, the same clinical information repository A may be accessed by two different network paths 1 and 2. Similarly, in FIGS. 2B and 2D the same clinical information repository B may be accessed by the two different network paths 1 and 2. The same network path 1 may be used to access different clinical information repositories A and B as shown in FIGS. 2A and 2B and the same network path 2 may be used to access different clinical information repositories A and B as shown in FIGS. 2C and 2D. The characteristic values of the parameters associated with a network source may be based on the network path aspect of the network source and/or the clinical information repository aspect of the network path.

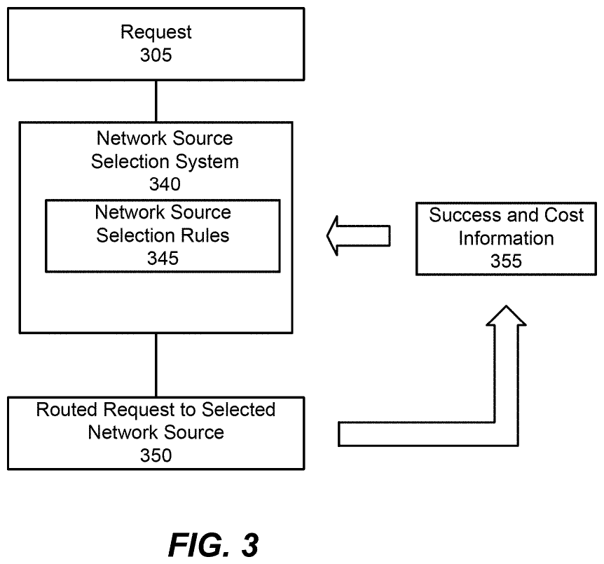

FIG. 3 is a block diagram that illustrates embodiments of the network source selection system for retrieving clinical information in which rules are applied to evaluate the request and the associated requested values for one or more parameters associated with the network sources to make the network source selection. Referring to FIG. 3, a request 305 is provided to the network source selection system 340. The network source selection system is configured with network source selection rules, which are used compare the requested values for the one or more parameters associated with the network sources that is associated with the request against the characteristic values for these parameters of the respective network sources. As part of the comparison, the network source selection rules may place greater emphasis on some parameters over others based on priority weights that may be defined by the requestor, for example. Based on the comparison, the network source selection rules 345 may be configured to select one of the network sources to be used for fulfilling the request. The request may then be routed to the selected network source 350 for fulfillment. The network source selection system 340 may determine whether the request was fulfilled successfully and may also determine the financial cost associated with fulfilling the request 355. This information may then be used to update the characteristic values for the parameters corresponding to the selected network source. For example, if the network source has a financial cost parameter characteristic value of $3 per record pull, but the network source charged $5 per record pull for this request, then the financial cost parameter characteristic value for this network source many be increased to $3.75 representing an average cost per record pull.

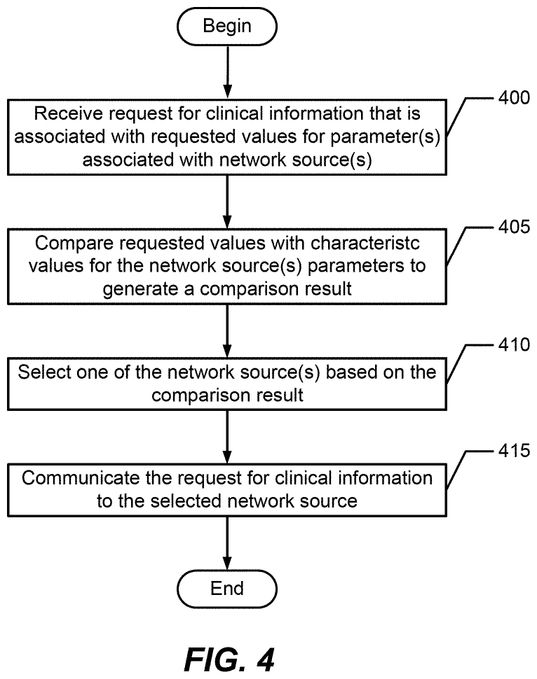

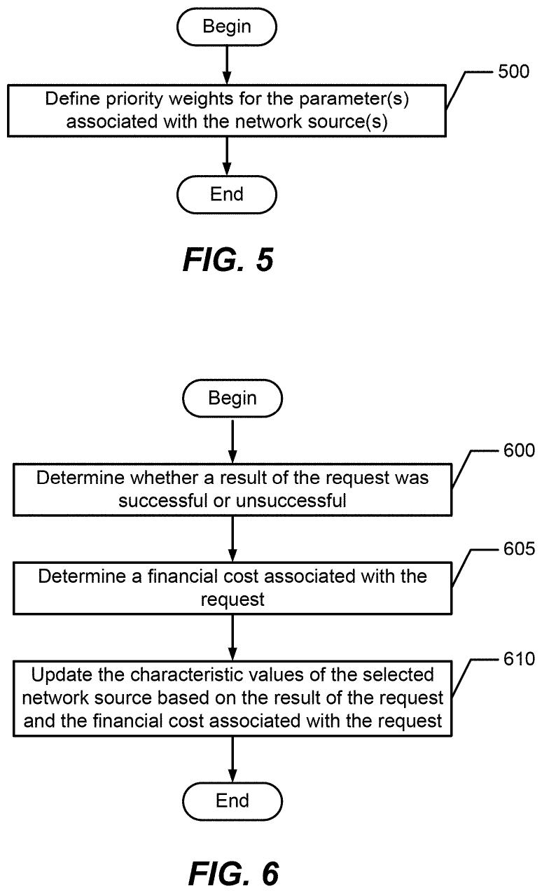

FIGS. 4-6 are flowcharts that illustrate operations for automatically selecting a network source for retrieving clinical information in accordance with some embodiments of the inventive concept. Referring now to FIG. 4, operations begin at block 400 where a request is received for clinical information. The request is associated with requested values for one or more parameters associated with network sources for retrieving the clinical information. The parameters may include, but are not limited to, a financial cost for fulfilling the request, a success rate in fulfilling requests, a media type used in fulfilling requests (e.g., electronic format or paper format), a media access type used in fulfilling requests (e.g., use of the Fast Healthcare Interoperability Resources (FHIR) protocol, use of the Electronic Data Interchange (EDI) format, use of the United States Core Data for Interoperability (USCDI) Consolidated-Clinical Document Architecture (C-CDA) format, use of paper communication and/or electronic mail messaging), a response time for request fulfillment, a document processing technique applied for request fulfillment, and the purposes of use supported in fulfilling requests. In accordance with various embodiments of the inventive concept, the requested values for the one or more parameters may be obtained in a variety of different ways including, but not limited to, included with the request for the clinical information, agreed upon in advance, such as through a service level agreement, business rule, contract, or the like, and/or accessed through a stored requestor profile. At block 405, a comparison of the requested values for the one or more parameters associated with the network sources may be made with characteristic values for the one or more parameters of the different network sources to generate a comparison result. Referring to FIG. 5, priority weights may defined at block 500 for the various parameters associated with the network sources expressing a requestor's preferences regarding the relative importance of one parameter to another. Thus, at block 410, one of the network sources may be selected based on the comparison result and the priority weights assigned to the one or more parameters associated with the network sources. The request may then be communicated to the selected network source to retrieve the clinical information for the requestor at block 415.

As described above, the network source selection system, according to some embodiments of the inventive concept, may be configured to monitor the result of the request to update the characteristic values for parameters corresponding to the selected network source. Referring to FIG. 6, a determination is made whether the result of the request was successful or unsuccessful at block 600. A determination of the financial cost associated with fulfilling the request is determined at block 605. The success information determined at block 600 and the cost information determined at block 605 may then be used to update the characteristic values for the success rate for request fulfillment and financial cost for request fulfillment parameters corresponding to the selected network source at block 610. Thus, a network source's reliability as reflected in its characteristic value for the success rate for request fulfillment parameter and cost as reflected in its characteristic value for the financial cost for request fulfillment parameter may be dynamically maintained based on the results of each request communicated to the network source.

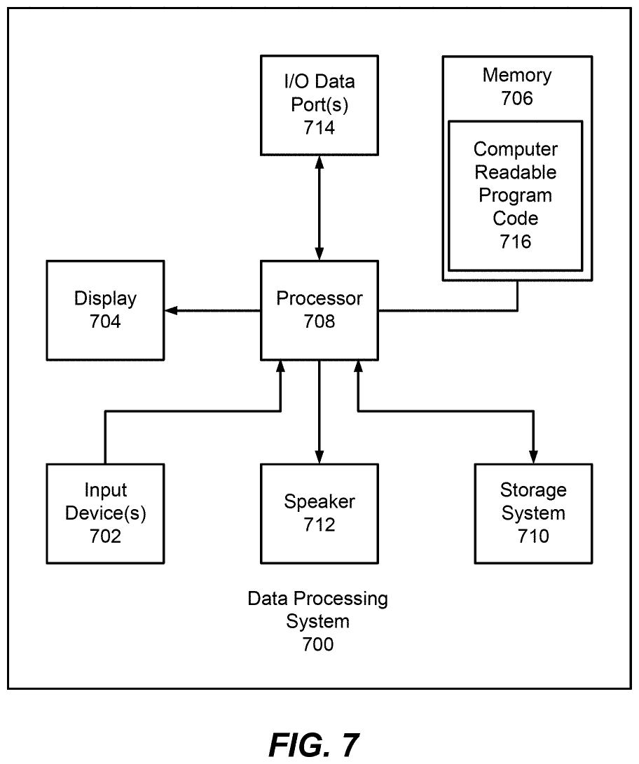

Referring now to FIG. 7, a data processing system 700 that may be used to implement the network source selection server 130 of FIG. 1 and the network source selection system 340 of FIG. 3, in accordance with some embodiments of the inventive concept, comprises input device(s) 702, such as a keyboard or keypad, a display 704, and a memory 706 that communicate with a processor 708. The data processing system 700 may further include a storage system 710, a speaker 712, and an input/output (I/O) data port(s) 714 that also communicate with the processor 708. The processor 708 may be, for example, a commercially available or custom microprocessor. The storage system 710 may include removable and/or fixed media, such as floppy disks, ZIP drives, hard disks, or the like, as well as virtual storage, such as a RAMDISK. The I/O data port(s) 714 may be used to transfer information between the data processing system 700 and another computer system or a network (e.g., the Internet). These components may be conventional components, such as those used in many conventional computing devices, and their functionality, with respect to conventional operations, is generally known to those skilled in the art. The memory 706 may be configured with computer readable program code 716 to facilitate the selection of a network source for retrieval of clinical information in accordance with some embodiments of the inventive concept.

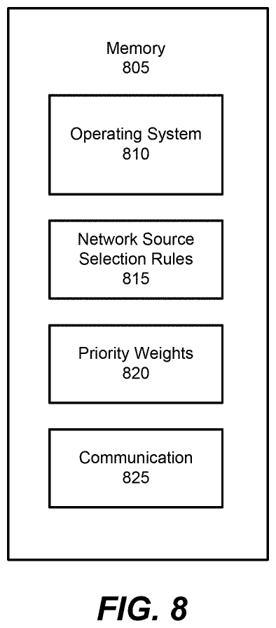

FIG. 8 illustrates a memory 805 that may be used in embodiments of data processing systems, such as the network source selection server 130 of FIG. 1, the network source selection system 340 of FIG. 3, and the data processing system of FIG. 7, respectively, to facilitate selection of a network source for retrieval of clinical information. The memory 805 is representative of the one or more memory devices containing the software and data used for facilitating operations of the network source selection server 130 and the network source selection module 135 as described herein. The memory 805 may include, but is not limited to, the following types of devices: cache, ROM, PROM, EPROM, EEPROM, flash, SRAM, and DRAM. As shown in FIG. 8, the memory 805 may contain four or more categories of software and/or data: an operating system 810, network source selection rules module 815, priority weights 820, and a communication module 835. In particular, the operating system 810 may manage the data processing system's software and/or hardware resources and may coordinate execution of programs by the processor.

The network source selection rules module 815 may provide the rules 345 for automatically selection a network source for retrieving clinical information as described above with respect to FIG. 3. The priority weights 820 may provide the priority weights defined, for example, by requestors for one or more parameters associated with the network sources as described above with respect to FIG. 5, for example. The communication module 825 may be configured to facilitate communication between the network source selection server 130 of FIG. 1 and/or the network source selection system 340 of FIG. 3 and the requestors 160a, 160b, and 160c of FIG. 1 along communication between the network source selection server 130 of FIG. 1 and/or the network source selection system 340 of FIG. 3 and the network sources for the retrieving the clinical information.

Although FIGS. 7 and 8 illustrate hardware/software architectures that may be used in data processing systems, such as the network source selection server 130 of FIG. 1, the network source selection system 340 of FIG. 3, and the data processing system of FIG. 7, respectively, in accordance with some embodiments of the inventive concept, it will be understood that the present invention is not limited to such a configuration but is intended to encompass any configuration capable of carrying out operations described herein.

Computer program code for carrying out operations of data processing systems discussed above with respect to FIGS. 1-8 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the network source selection server 130 of FIG. 1, the network source selection system 340 of FIG. 3, and the data processing system of FIG. 7 may each be implemented as a single processor system, a multi-processor system, a multi-core processor system, or even a network of stand-alone computer systems, in accordance with various embodiments of the inventive concept. Each of these processor/computer systems may be referred to as a “processor” or “data processing system.”

The data processing apparatus described herein with respect to FIGS. 1-8 may be used to facilitate automatically selecting a network source for retrieving clinical information according to some embodiments of the inventive concept described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 805 when coupled to a processor includes computer readable program code that, when executed by the processor, causes the processor to perform operations including one or more of the operations described herein with respect to FIGS. 1-6.

Some embodiments of the inventive concept may provide a network source selection system that may automate the evaluation of multiple network sources for retrieving clinical information for a requestor thereby improving the ability of a requestor to obtain clinical information a more cost effective and efficient manner. Moreover, a requestor may assign priority weights to parameters associated with the network sources to express the requestor's preferences with respect to the relative importance of one parameter to another. This may allow the network source selection system to select a network source for fulfilling a clinical information request that more closely aligns with the requestor's preferences with respect to cost, format, speed, reliability, or other factor.

### Further Definitions and Embodiments

In the above-description of various embodiments of the present inventive concept, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

The flowchart and block diagrams in the figures illustrate the architecture, functionality, and operation of possible implementations of systems, methods, and computer program products according to various aspects of the present inventive concept. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

In the above-description of various embodiments of the present inventive concept, aspects of the present inventive concept may be illustrated and described herein in any of a number of patentable classes or contexts including any new and useful process, machine, manufacture, or composition of matter, or any new and useful improvement thereof. Accordingly, aspects of the present inventive concept may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present inventive concept may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

The description of the present inventive concept has been presented for purposes of illustration and description, but is not intended to be exhaustive or limited to the inventive concept in the form disclosed. Many modifications and variations will be apparent to those of ordinary skill in the art without departing from the scope and spirit of the inventive concept. The aspects of the inventive concept herein were chosen and described to best explain the principles of the inventive concept and the practical application, and to enable others of ordinary skill in the art to understand the inventive concept with various modifications as are suited to the particular use contemplated.

What is claimed is:

1. A method, comprising:
receiving a request for clinical information associated with one or more patients from a requestor, the request being associated with requested values for one or more parameters associated with a plurality of network sources for retrieving the clinical information;
automatically comparing the requested values with characteristic values for the one or more parameters of each of the plurality of network sources to generate a comparison result;
automatically selecting one of the plurality of network sources based on the comparison result; and
communicating the request for clinical information to the one of the plurality of network sources that was selected.

2. The method of claim 1, wherein at least one of the requested values is received with the request for the clinical information.

3. The method of claim 1, further comprising:
determining whether a result of the request that was communicated to the one of the plurality of network sources was successful or unsuccessful;
determining a financial cost associated with the request that was communicated to the one of the plurality of network sources; and
updating the characteristic values of the one of the plurality of network sources based on the result of the request and the financial cost associated with the request.

4. The method of claim 1, wherein the one or more parameters associated with the plurality of network sources comprise:
a financial cost for request fulfillment;
a success rate for request fulfillment;
a media type used for request fulfillment;
a media access type used for request fulfillment;
a response time for request fulfillment;
a document processing technique applied for request fulfillment; or
purposes of use supported for request fulfillment.

5. The method of claim 4, wherein the media type used for request fulfillment parameter is configured to assume a value of electronic format or paper format.

6. The method of claim 4, wherein the media access type used for request fulfillment parameter is configured to assume one or more values of Fast Healthcare Interoperability Resources (FHIR) protocol, Electronic Data Interchange (EDI) format, United States Core Data for Interoperability (USCDI) Consolidated-Clinical Document Architecture (C-CDA) format, paper communication and/or electronic mail messaging.

7. The method of claim 4, wherein the financial cost for request fulfillment is configured to be expressed as a financial cost for a single request for clinical information for a single patient; and/or
wherein the financial cost is configured to be expressed as a financial cost for multiple requests for clinical information of a single patient over a defined time period.

8. The method of claim 7, wherein the defined time period is one year or one month.

9. The method of claim 4, wherein the financial cost for request fulfillment is dependent on values for the media type used for request fulfillment parameter and/or the media access type used for request fulfillment parameter.

10. The method of claim 4, wherein the purposes of use supported for request fulfillment is configured to assume a value of benefits determination, life insurance eligibility determination, clinical research, or patient risk adjustment for insurance.

11. The method of claim 1, further comprising:
defining priority weights for the one or more parameters associated with the plurality of network sources;
wherein automatically selecting the one of the plurality of network sources based on the comparison result comprises automatically selecting the one of the plurality of network sources based on the comparison result and the priority weights for the one or more parameters associated with the plurality of network sources.

12. A system, comprising:
a processor; and
a memory coupled to the processor and comprising computer readable program code embodied in the memory that is executable by the processor to perform operations comprising:
receiving a request for clinical information associated with one or more patients from a requestor, the request being associated with requested values for one or more parameters associated with a plurality of network sources for retrieving the clinical information;
automatically comparing the requested values with characteristic values for the one or more parameters of each of the plurality of network sources to generate a comparison result;
automatically selecting one of the plurality of network sources based on the comparison result; and
communicating the request for clinical information to the one of the plurality of network sources that was selected.

13. The system of claim 12, wherein the operations further comprise:
determining whether a result of the request that was communicated to the one of the plurality of network sources was successful or unsuccessful;
determining a financial cost associated with the request that was communicated to the one of the plurality of network sources; and
updating the characteristic values of the one of the plurality of network sources based on the result of the request and the financial cost associated with the request.

14. The system of claim 12, wherein the one or more parameters associated with the plurality of network sources comprise:
a financial cost for request fulfillment;
a success rate for request fulfillment;
a media type used for request fulfillment;
a media access type used for request fulfillment;
a response time for request fulfillment;
a document processing technique applied for request fulfillment; or
purposes of use supported for request fulfillment;
wherein the media type used for request fulfillment parameter is configured to assume a value of electronic format or paper format;
wherein the media access type used for request fulfillment parameter is configured to assume one or more values of Fast Healthcare Interoperability Resources (FHIR) protocol, Electronic Data Interchange (EDI) format, United States Core Data for Interoperability (USCDI) Consolidated-Clinical Document Architecture (C-CDA) format, paper communication and/or electronic mail messaging;
wherein the financial cost for request fulfillment is configured to be expressed as a financial cost for a single request for clinical information for a single patient, and/or wherein the financial cost is configured to be expressed as a financial cost for multiple requests for clinical information of a single patient over a defined time period;
wherein the financial cost for request fulfillment is dependent on values for the media type used for request fulfillment parameter and/or the media access type used for request fulfillment parameter; and
wherein the purposes of use supported for request fulfillment is configured to assume a value of benefits determination, life insurance eligibility determination, clinical research or patient risk adjustment for insurance.

15. The system of claim 14, wherein the defined time period is one year or one month.

16. The system of claim 12, wherein the operations further comprise:
defining priority weights for the one or more parameters associated with the plurality of network sources;
wherein automatically selecting the one of the plurality of network sources based on the comparison result comprises automatically selecting the one of the plurality of network sources based on the comparison result and the priority weights for the one or more parameters associated with the plurality of network sources.

17. A computer program product, comprising:
a non-transitory computer readable storage medium comprising computer readable program code embodied in the medium that is executable by a processor to perform operations comprising:
receiving a request for clinical information associated with one or more patients from a requestor, the request being associated with requested values for one or more parameters associated with a plurality of network sources for retrieving the clinical information;
automatically comparing the requested values with characteristic values for the one or more parameters of each of the plurality of network sources to generate a comparison result;
automatically selecting one of the plurality of network sources based on the comparison result; and
communicating the request for clinical information to the one of the plurality of network sources that was selected.

18. The computer program product of claim 17, wherein the operations further comprise:
determining whether a result of the request that was communicated to the one of the plurality of network sources was successful or unsuccessful;
determining a financial cost associated with the request that was communicated to the one of the plurality of network sources; and
updating the characteristic values of the one of the plurality of network sources based on the result of the request and the financial cost associated with the request.

19. The computer program product of claim 17, wherein the one or more parameters associated with the plurality of network sources comprise:
a financial cost for request fulfillment;
a success rate for request fulfillment;
a media type used for request fulfillment;
a media access type used for request fulfillment;
a response time for request fulfillment;
a document processing technique applied for request fulfillment; or
purposes of use supported for request fulfillment;
wherein the media type used for request fulfillment parameter is configured to assume a value of electronic format or paper format;
wherein the media access type used for request fulfillment parameter is configured to assume one or more values of Fast Healthcare Interoperability Resources (FHIR) protocol, Electronic Data Interchange (EDI) format, United States Core Data for Interoperability (USCDI) Consolidated-Clinical Document Architecture (C-CDA) format, paper communication and/or electronic mail messaging;
wherein the financial cost for request fulfillment is configured to be expressed as a financial cost for a single request for clinical information for a single patient, and/or wherein the financial cost is configured to be expressed as a financial cost for multiple requests for clinical information of a single patient over a defined time period;
wherein the financial cost for request fulfillment is dependent on values for the media type used for request fulfillment parameter and/or the media access type used for request fulfillment parameter; and
wherein the purposes of use supported for request fulfillment is configured to assume a value of benefits determination, life insurance eligibility determination, clinical research, or patient risk adjustment for insurance.

20. The computer program product of claim 19, wherein the defined time period is one year or one month.

21. The computer program product of claim 17, wherein the operations further comprise:
defining priority weights for the one or more parameters associated with the plurality of network sources;
wherein automatically selecting the one of the plurality of network sources based on the comparison result comprises automatically selecting the one of the plurality of network sources based on the comparison result and the priority weights for the one or more parameters associated with the plurality of network sources.
